(19) United States
(12) Patent Application Publication
(10) Pub. No.: US 2025/0025040 A1
(43) Pub. Date: Jan. 23, 2025

(54) METHODS, SYSTEMS, AND COMPUTER READABLE MEDIA FOR BIOMETRIC OCULAR PHOTOMETRY

(71) Applicant: The University of North Carolina at Chapel Hill, Chapel Hill, NC (US)

(72) Inventors: Nicolas Christian Richard Pégard, Chapel Hill, NC (US); Jose Manuel Rodriguez, Chapel Hill, NC (US); Therese Marie Curtis, Chapel Hill, NC (US); Maria Magdalena Ortiz, Chapel Hill, NC (US); Mohammad Hossein Eybposh, Chapel Hill, NC (US); Tal Cohen, Chapel Hill, NC (US); Leon Geronimo Pavel Velazquez Hernandez, Chapel Hill, NC (US)

(21) Appl. No.: 18/713,626

(22) PCT Filed: Dec. 8, 2022
(86) PCT No.: PCT/US2022/052302

§ 371 Date: May 24, 2024

Related U.S. Application Data
(60) Provisional application No. 63/287,482, filed on Dec. 8, 2021.

(51) Int. Cl. A61B 3/00; A61B 3/13; A61B 5/00; A61B 5/0205; A61B 5/11; A61B 5/16

(52) U.S. Cl. CPC A61B 3/0008; A61B 3/13; A61B 5/0086; A61B 5/0205; A61B 5/163; A61B 5/6803; A61B 5/1103; A61B 2503/40; A61B 2562/0238

(57) ABSTRACT

A system for determining a physical characteristic or measuring a physical activity of a subject using back-illumination of an eye of a subject includes at least one light source configured for illuminating an eye of a subject using light from within the head of the subject. The system further includes at least one light sensor positionable outside of the head of the subject for sensing light from the at least one light source exiting the subject through the eye of the subject. The system further includes a controller coupled to the at least one light source and to the at least one light sensor for recording an indication of the light while controlling the illuminating.

### PRIORITY CLAIM

This application claims the priority benefit of U.S. Provisional Patent Application Ser. No. 63/287,482, filed Dec. 8, 2021, the disclosure of which is incorporated herein by reference in its entirety.

### TECHNICAL FIELD

The subject matter described herein relates to determining physical characteristics and actions of a subject. More particularly, the subject matter described herein relates to determining physical characteristics and actions of a subject using illumination from behind the eye, also referred to herein as back-illumination of the subject's eye.

### BACKGROUND

Biometric monitoring is the observation of conscious and unconscious physiological responses to stimuli. Tracking pupil size and location, respiratory, and heart rate dynamics are key measurements that altogether characterize physiological arousal responses. Pupillometry is a non-invasive technique that measures pupil size and position over time and traditionally relies on a camera to capture videos of the pupil illuminated externally with infrared (IR) light⁷⁻⁹. This approach requires bulky equipment placed near the eye that occludes the field of view, preventing subjects from performing the full repertoire of natural behaviors¹¹. Extracting pupil size dynamics from the video data requires computationally expensive segmentation algorithms to be applied to each frame. Sampling rates are limited by the available camera frame rate, typically at or below 100 fps, preventing the capture of high speed physiological events, and real-time operation is computationally challenging. Certain gaze tracking and pupillometry systems replace the camera in favor of infrared emitters and sensors placed in front of the eyes¹⁰, however these systems do not segregate pupillary responses arising from arousal changes from those caused by fluctuations in the ambient light.

The pupillary light reflex is an important metric of autonomic nervous system function that has been exploited for a wide range of clinical applications to diagnose multiple neurological disorders². However, these measurements are taken across brief periods of time (seconds) and longitudinal tracking of pupillary light responses across longer periods of time (minutes, hours, days, months) have not been studied. Furthermore, these systems are limited to measuring pupil dynamics while heart and respiratory biometrics must be acquired using stress inducing neck collars¹² or invasive methods that require surgical implantation of a device inside the body¹³. To capture all relevant biometrics, several separate and often expensive devices must measure signals from the subject simultaneously, leading to noise, errors in data synchronization, and an increased cost for the user. In light of these and other difficulties, there exists a need for improved methods, systems, and computer readable media for determining physical characteristics and actions of a subject using illumination of the eye(s) of the subject.

### SUMMARY

A system for determining a physical characteristic or action of a subject using internal illumination of the back of an eye of a subject includes at least one light source configured for illuminating an eye of a subject using light from within a head of the subject. The system includes at least one light sensor positionable outside of the head of the subject for sensing light from the at least one light source exiting the subject through the eye of the subject. The system further includes a controller coupled to the at least one light source and at least one light sensor for recording an indication of the light while controlling the illuminating.

According to another aspect of the subject matter described herein, the at least one light source comprises an infrared light source.

According to another aspect of the subject matter described herein, the infrared light source comprises an infrared emitter circuit configured to generate pulses of infrared light.

According to another aspect of the subject matter described herein, the infrared emitter circuit is configured to generate continuous illumination of pulses of light at instantaneous power levels ranging from 1 milliwatt to 10 Watts.

According to another aspect of the subject matter described herein, the at least one light source comprises a housing configured to be inserted into an ear canal or a nostril of the subject or positioned adjacent to a surface of the skin of the subject.

According to another aspect of the subject matter described herein, the at least one light source comprises an implantable device for implantation within the head of the subject.

According to another aspect of the subject matter described herein, the at least one light source comprises a primary light source for illuminating the eye of the subject from within the head of the subject and a secondary light source for controlling a pupil of the eye of the subject by illuminating the pupil externally to the head of the subject.

According to another aspect of the subject matter described herein, secondary light source comprises a visible light source.

According to another aspect of the subject matter described herein, the system includes a head mountable frame for holding the at least one light source.

According to another aspect of the subject matter described herein, the head mountable frame is configured to hold the at least one light sensor.

According to another aspect of the subject matter described herein, the system includes a mirror for directing the light exiting the subject through the eye of the subject to the at least one light sensor.

According to another aspect of the subject matter described herein, the at least one light source comprise an infrared light source and the mirror comprises an infrared mirror.

According to another aspect of the subject matter described herein, the at least one light sensor comprises a light sensor array including a plurality of infrared receiver circuits positionable around an exterior of the eye of the subject.

According to another aspect of the subject matter described herein, the infrared receiver circuits each comprise an optoelectronic device for detecting the light and generating a current proportional to the detected light.

According to another aspect of the subject matter described herein, the optoelectronic device comprises a photodetector.

According to another aspect of the subject matter described herein, the system includes a head mountable frame for holding at least one light sensor on the head of the subject.

According to another aspect of the subject matter described herein, the head mountable frame has a glasses-like or goggles-like form factor.

According to another aspect of the subject matter described herein, the head mountable frame comprises an annulus for holding the at least one light sensor near the eye of the subject.

According to another aspect of the subject matter described herein, the controller includes an analog-to-digital converter for producing digital values based on a signal generated by the at least one light sensor.

According to another aspect of the subject matter described herein, the controller records at least one signal generated by the at least one light sensor while synchronously controlling the at least one light source to generate pulses of light.

According to another aspect of the subject matter described herein, the system includes a biometric measurement module for determining a physical characteristic of the subject based on the light sensed by the at least one light sensor.

According to another aspect of the subject matter described herein, the biometric measurement module is configured to determine at least one of eye movement, eye blinking, and pupil size variation based on the light sensed by the at least one light sensor.

According to another aspect of the subject matter described herein, the biometric measurement module is configured to determine at least one of heart rate, heart beats, blood flow, pulse oximetry, breathing rate, and respirations of the subject based on the light sensed by the at least one light sensor.

According to another aspect of the subject matter described herein, the system includes a calibration module that implements an algorithm configured to calibrate the biometric measurement module.

According to another aspect of the subject matter described herein, the controller is configured to subtract a representation of a signal generated by the at least one light sensor when the at least one light source is off from a representation of the signal generated by the at least one light sensor when the at least one light source is on for measuring the amount of light exiting the eye regardless of ambient light levels.

According to another aspect of the subject matter described herein, the at least one light sensor is configured to sense light exiting the subject through a pupil of the eye of the subject.

According to another aspect of the subject matter described herein, a method for determining a physical characteristic or measuring an activity of a subject using back-illumination of an eye of the subject is provided. The method includes positioning at least one light source for illuminating an eye of a subject using light from within a head of the subject. The method further includes controlling the at least one light source to illuminate the eye of the subject from within the head of the subject. The method further includes sensing, using at least one light sensor located external to the head of the subject, light from the at least one light source exiting the subject through the eye of the subject. The method further includes recording an indication of the light sensed by the at least one light sensor while controlling the illuminating.

According to another aspect of the subject matter described herein, positioning the at least one light source includes positioning the at least one light source in an ear canal, in a nostril, or adjacent to a surface of the skin of the subject.

According to another aspect of the subject matter described herein, positioning the at least one light source includes positioning the at least one light source within or on the head of the subject.

According to another aspect of the subject matter described herein, recording the indication of the light sensed by the at least one sensor while controlling the illuminating includes recording at least one signal generated by the at least one sensor while synchronously controlling the at least one light source to generate pulses of light.

According to another aspect of the subject matter described herein, the method includes determining a physical characteristic or action of the subject based on the light sensed by the at least one light sensor.

According to another aspect of the subject matter described herein, determining the physical characteristic or measuring the activity includes measuring at least one of eye movement, eye blinking, and pupil size variation based on the light sensed by the at least one light sensor.

According to another aspect of the subject matter described herein, the method includes determining a physical characteristic or activity of the subject based on the light sensed by the at least one light sensor.

According to another aspect of the subject matter described herein, the physical characteristic includes at least one of eye movement, eye blinking, and pupil size variation based on the light sensed by the at least one light sensor.

According to another aspect of the subject matter described herein, the physical characteristic or activity includes at least one of heart rate, heart beats, blood flow, pulse oximetry, breathing rate, and respirations of the subject based on the light sensed by the at least one light sensor.

According to another aspect of the subject matter described herein, the method includes subtracting a representation of a signal generated by the at least one light sensor when the at least one light source is off from a representation of the signal generated by the at least one sensor when the at least one light source is on for measuring the amount of light exiting the pupil regardless of ambient light levels.

According to another aspect of the subject matter described herein, sensing the light exiting the eye of the subject includes light exiting the subject through a pupil of the eye of the subject.

According to another aspect of the subject matter described herein, a non-transitory computer readable medium having stored thereon executable instructions that when executed by a processor of a computer controls the computer to perform steps is provided. The steps included controlling at least one light source to illuminate the eye of the subject from within the head of the subject. The steps further include sensing, using at least one light sensor located external to the head of the subject, light from the at least one light source exiting the subject through the eye of the subject. The steps further include recording an indication of the light sensed by the at least one light sensor while controlling the illuminating.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Exemplary implementation of the subject matter described herein will now be explained with reference to the accompanying drawings, of which:

FIG. 1 is a schematic diagram illustrating exemplary components of a system for infrared ocular back-illumination;

FIG. 2A is a diagram of the head of a mouse, an infrared light source, and a photodetector back-illumination of the eye of the mouse and detection of the resulting light exiting the eye;

FIG. 2B is a diagram of the head of a human, different possible positionings of an infrared light source for back-illumination of the human's eyes, and a photodetector array mounted on a frame having a glasses-like form factor.

The FIG. 3A is a diagram of the head of a mouse and a biometric optical photometer including an infrared LED and a photodiode for back-illuminating one of the eyes of the mouse and detecting infrared light exiting one pupil of the mouse. FIG. 3A also illustrates an infrared camera for recording images of a pupil of the mouse. FIG. 3A further includes a diagram of a segmented pupil of the mouse generated from images produced by the infrared camera.

FIG. 3B is a diagram of a mouse, a photodiode for detecting light exiting the pupils of the mouse, electrocardiogram (EKG) leads, and a piezo sensor;

FIG. 3C is a graph of a pupil size signal generated from the signal produced by the biometric optical photometer of FIG. 3A and a ground truth signal generated from the pupil images recorded by the IR camera in FIG. 3A;

FIG. 3D is a graph illustrating a heart beat signal generated from the signal produced by the biometric optical photometer of FIG. 3A and a ground truth signal produced using an EKG whose leads are illustrated in FIG. 3B;

FIG. 3E is a graph illustrating a respiration signal generated from the signal produced by the biometric optical photometer of FIG. 3A and a ground truth signal produced using the piezo sensor in FIG. 3B;

FIG. 4 is a graph illustrating near-eye detection of diffused IR light using one photodetector of the photodetector array illustrated in FIG. 2B. The results show increased IR light flow to the photodetector when looking to the left side where the IR photodetector is placed;

FIG. 5 is a circuit diagram illustrating exemplary components of an IR emitter circuit, an IR receiver circuit, and a controller;

FIGS. 6A and 6B are diagrams illustrating an example of a housing for holding light sources and light sensors where the housing is a head-mountable device designed to be worn on the head of a subject;

FIG. 7 is a diagram of a housing designed to hold a light sensor where the housing is designed to be insertable and worn in the ear canal of the subject;

FIGS. 8A and 8B are diagrams of a housing designed to hold light detectors near the eyes of a subject where the housing comprises a pair of goggles; and

FIG. 9 is a flow chart illustrating an exemplary process for determining a physical characteristic or activity of a subject using back-illumination of the subject's eye.

### DETAILED DESCRIPTION

We have developed a lightweight low-cost technology for recording multiple biometric indicators of arousal simultaneously, continuously, and in real-time. The subject matter described herein includes a device that comprises a low-power, pulsed source of infrared light that illuminates the back of the eye from behind it, with infrared light entering the body from the back or the side, or inside the brain (in animals) that diffuses through tissue, bone and skin. Pulsed illumination is synchronized with photodetectors placed near the eye that continuously measure the amount of diffused infrared light exiting through the pupil across multiple directions. The subject matter described herein includes custom computational methods to acquire the raw data and process the data to extract quantitative measurements of ocular biometrics and other physical characteristics of a subject. The subject matter described can measure pupil size and direction (eye movement), heart and breathing rates, the ambient light level, and head motion, simultaneously, in real-time, and at multi-kilohertz sampling speeds. Our technology is safe to be utilized both in animals and humans, and can be built with inexpensive, lightweight, and low-power electronics.

We anticipate that our technology will be of interest to (1) the scientific community, for the study and development of treatment models for neurological and neuropsychiatric disorders affecting arousal, mood, sleep, and attention. We also foresee (2) clinical and medical applications since many neurological disorders have symptomatology that affect autonomic properties. Here, our technology can facilitate the early detection of neurological disorders, of adverse events and symptoms, and help address them as needed with precisely timed treatments. In addition, it can be used to detect onset of hypo- and hyper-arousal state changes in patients suffering from neuropsychiatric disorders related to mood and anxiety. Our technology is also perfectly suited for (3) consumer-level applications such as video games, advertisement, and the broader self-care industry where the ability to monitor biometrics in real-time can provide feedback to optimize content and/or to validate or enhance the desired user's reaction. Finally, our technology can be implemented in (4) smart personal protection equipment to track warning signs of fatigue, attention to tasks, and help prevent human errors in critical situations.

Biometric monitoring is the observation of conscious and unconscious physiological responses to stimuli. Tracking pupil size and location, respiratory, and heart rate dynamics are key measurements that altogether characterize physiological arousal responses. Pupillometry is a non-invasive technique that measures the eye's pupil size and position over time¹. Variations of the pupil size happen, as a reflexive process in response to variations in the surrounding light level, but also through a voluntary mechanism driven by physiological arousal and higher-level cognition. For example, positive arousal and cognitively demanding tasks generally lead to an increase in pupil size. Hence, rapid pupillary responses are a reliable indicator of changes in emotional and attentional states. Measuring pupil size fluctuations provides valuable information about how mammalian species interact with their surrounding environments. Therefore, pupillometry data provides important insights for research across human and preclinical animal models of health and disease.

There is a growing body of literature indicating that abnormal pupil responses occur in patients experiencing neurological and neuropsychiatric disorders¹⁻³. Studies have highlighted that abnormal pupil responses can be early indicators of neurological diseases such as Parkinson's, Autism, and Multiple Sclerosis⁴. Early detection of neurological disease is at the forefront of biomedical research studies to improve patient outcomes through prompt medical intervention. Detecting adverse neurological events is also highly valuable in clinical settings to improve treatment plans with a precisely timed intervention, for instance in combination with the delivery of fast acting drugs. Our technique could be very useful for patients suffering from neuropsychiatric disorders where both patient and clinician can detect early phases of hypo- or hyper-arousal states before they escalate to either a panic attack or suicide.

One of the main advantages of the pupillometry technique we propose is that our system is non-invasive, and portable. This allows any user to perform longitudinal studies, and gather data without disturbing the animal or patient for extended periods. Also, our technology can be used across several species without any substantial hardware modifications which facilitates translational research. Pupillometry has also been implemented to improve the performance of brain-computer interfaces for patients that have deficits in motor control and are nonverbal. Utilizations include locked-in-patients (pseudocoma) that rely on clinicians and family members to monitor pupil movement towards letters to communicate. Recently, one study found that using changes in pupil diameter that occur when eyes are focused on letters leads to more accurate communication from locked-in-patients compared to only using eye movement5. This indicates that our technology can answer a need for real-time pupillometry technology to reliably track pupil responses and enhance the performance of brain-computer interfaces.

Additionally, the use of pupillometry data extends beyond basic research and clinical applications. Since changes in pupil response occur when a subject is performing mental challenging tasks, one study used pupillometry as a way to measure difficulty levels of educational video games amongst students6. This information was helpful for instructors to assess student learning. Pupillometry can also provide feedback in video games or other forms of entertainment, to optimize the user's experience. Our technology is also useful to gather feedback with study groups when evaluating the effectiveness of advertisement campaigns. Finally, our technology can be integrated into safety goggles, to create smart monitors that track sleep or loss of attention and reduce accidents caused by human errors in job sites where personal safety is critical.

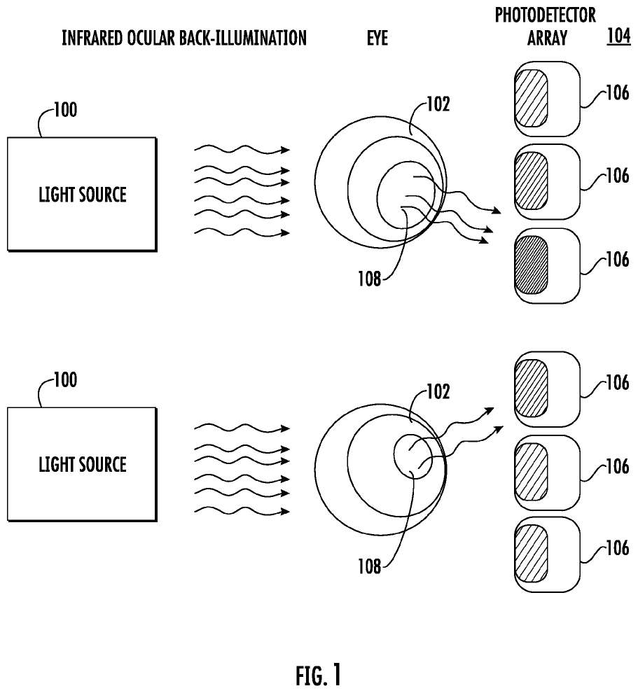

The subject matter described herein includes a portable device that measures the size and location of the pupil with photodetectors placed near the eye. As opposed to existing techniques that illuminate the front of the eye with light to capture pupil information, our approach relies on a back-illumination method with infrared light diffusing through bones and tissue to illuminate the back of the eye (FIG. 1).

More particularly, FIG. 1 illustrates pupil tracking with multiple photodetectors capturing diffuse infrared light through the pupil. In FIG. 1, light from one or more light sources 100 back-illuminates eyes 102 of a subject. A sensor array 104 including a plurality of individual light sensors 106 senses light exiting the subject through pupils 108 of eyes 102. In the illustrated example, light sources 100 comprise infrared light sources. Light sources 100 may be positioned in or on the head of the subject to illuminate eyes 102 from within the head of the subject. In one example, light sources 100 each comprise an infrared emitter circuit (described below) located within a housing. If the subject is a human, the housing may be an insertable device configured to be inserted in the subject's ear canal or nostril. If the subject is a non-human subject, such as a non-human mammal, the housing may be an implantable device for implantation within the head of the subject.

The amount of light captured by an individual light sensor 106 increases when one of eyes 102 is pointed at the light sensor 106 and decreases when the eye 102 is pointed away from the light sensor 106. By mapping the amount of light captured by several of light sensors 106, and with a calibration step, we can estimate the eye orientation. By measuring the total amount of light captured by all of light sensors 106, we can then estimate the size of the pupil, independently of its orientation.

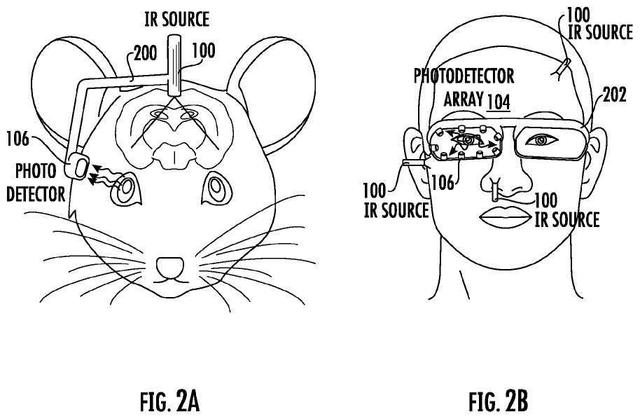

As indicated above, infrared (IR) illumination can be achieved by placing the source of IR light in the ear, nose, neck, or on the head and through the skull (FIGS. 2A and 2B). In animals, light can also be injected directly into the brain via an implanted optical fiber, or through a source placed flush with the skull or skin. Different IR wavelengths may be used in a rapid sequence for the acquisition of spectral information, to capture additional biometric variables such as blood oxygen saturation, as in pulse oximeters.

FIG. 2A illustrates exemplary components for determining physical characteristics or activities of subjects where the subject is a small mammal, such as a mouse. In FIG. 2A, light source 100 is an implantable device that injects IR light directly into the brain of the mouse through an implanted optical fiber, an implanted objective lens, or through surfaces of the skull/skin. In the illustrated example, light source 100 is connected to a light sensor 106 through a mechanical coupling 200. Light sensor 106 may be positioned near the animal's eye and pointed towards the eye to detect light exiting the eye through the pupil.

FIG. 2B illustrates examples where light sources can be used to back-illuminate the eyes of a human subject. In one example illustrated in FIG. 2B, an IR light source 100 includes a housing configured to be inserted within the ear canal of the human subject. In another example, an IR light source 100 includes a housing configured to be inserted within a nostril of the human subject. In another example, IR light source 100 may be positioned adjacent to the skin on the outside of the subject's head to back-illuminate the eyes using IR light that penetrates the skin, skull, and brain of the human subject. FIG. 2B also illustrates an example where photodetector array 104 includes a frame 202 with a glasses-like form factor, where individual light sensors 106 are circumferentially spaced around a periphery of the subject's eye. One or multiple photodetectors measure the intensity of diffused infrared light through the pupil. The amount of light collected by the photodetectors placed near the eye is modulated by several external factors that include:
1. Variations in ambient light (baseline)
2. Pupil size
3. Blood flow (disturbing the ability for light to diffuse through tissue)
4. Tissue motion (from locomotion and breathing)
5. Eye motion (If the pupil is closer to or away from photodetectors)
6. Blinking

The photodetectors of our device detect infrared light, which includes the IR light emitted by our illumination source, as well as IR light present in the ambient light. The flow of ambient light adds a positive baseline to our measurements of the IR photon flow through the pupil and fluctuates depending on head orientation and on the surrounding lighting environment. To separate ambient light from the detected signal, our technology pulses the IR illumination source. The detectors are synchronized with the pulsed light to capture one baseline data point when the IR light is off, directly followed by another datapoint when the IR light is on. This captures the baseline and the signal together. By subtracting the two sequentially recorded values we obtain a quantitative measurement of the signal, i.e. the flow of IR light emitted by the pulsed source and reaching each photodetector. The signal is a measurement of the pupil size and orientation, with fluctuations that are caused by the pulsed blood flow, and by head motion. The signal is further processed to extract ocular biometrics (2, 3, 4, 5, 6). The baseline is also recorded for each photodetector. The total amount of baseline signal recorded across all photodetectors is a quantitative measure of ambient light. Fluctuations of the ambient light are compensated by modulations of the pupil size that are needed to maintain a suitable range of brightness on the retina, but these modulations of the pupil size do not relate to the arousal or behavior. By identifying synchronous events between the ambient light level and the signal, our technique classifies changes in pupil size and identifies events that can be attributed to behavior and arousal independently of physiological pupil size adjustments that only occur to match variations of the ambient light levels.

Differential amounts of light across photodetectors in the baseline signal occurs when the subject changes location, which introduces local variations in the illumination environment perceived by each photodetector. Differential signals in the baseline will be computed to track subject motion, providing yet another indicator of arousal.

External factors (2, 6) all introduce modulations in the signal. To measure eye motion and detect blinking (5,6), either involuntary, or intentional (e.g., eye saccades, twitch, gaze tracking), we compare the amount of signal detected on each photodetector. The signal detected on any detector increases when the pupil is pointed towards it and decreases when pointing away from it (see FIG. 1). By comparing the amount of signal across several detectors placed at various known locations around the eye, and with a simple calibration step, we estimate the orientation of the eye, both laterally and vertically.

To quantify pupil size (2), we calculate the average amount of light captured by all the detectors around the eye. This quantity no longer depends on pupil location, but scales linearly with the surface of the pupil, with small perturbations induced by blood flow (3) and tissue motion (e.g. from breathing) (4).

To separate these quantities, we filter the pupil size data (2) sampled at kilohertz speeds with temporal band-pass filters at frequencies centered around predicted heart and breathing rates. The peak frequency in the filtered signal corresponds to the heart and breathing rates. Since our sampling speeds are significantly faster than all these events, the signal temporal resolution is sufficient to detect these events, even in the presence of noise.

Since our technology generates raw data that requires a significant amount of nonlinear processing, our invention also features a deep learning model to process raw data in real time and extract quantitative indicators of behavior and arousal (e.g., pupil size, pupil orientation, heart rate, heart beats breathing rate, respirations, subject motion, and ambient light).

For pulse oximetry through the eye, our system would be equipped with several IR illumination sources with distinct peak emission wavelengths, as in a finger-attached pulse oximeter. Here, our invention has the advantage of enabling measurements through the eye, and yields results that are not affected by skin color. Recent studies have shown that finger-clip pulse oximeters are more often likely to return false or misleading results when patients have dark skin color14. Our technology would avoid such issues. Our technology is scalable to enable use in both animals (FIG. 2A) and human subjects (FIG. 2B), either as a head-fixed or as a portable device. In animals (FIG. 2A), IR light can be delivered to the brain with an LED or with a laser source. The source is either external which is placed flush with the skull, behind the neck, inside the ear, or mouth. The source can also be internal, delivered deep into the brain through an implanted objective or optical fiber, which is a common surgical procedure used in many optogenetic protocols. An alternative option suitable both for animals and humans (FIG. 2B) is to deliver the IR light externally, either by placing an LED light in the back of the neck, on the skull, or into the ear or nose.

In head-fixed cases, the animal is restrained, and light is delivered through the brain to come out of the pupils. The IR light source in these embodiments is typically either an LED placed flush with the skull, or the laser diode is coupled with an optic fiber for easy integration with pre-existing optogenetic protocols (FIG. 2A). For freely moving small animal subjects, some implementations include features that prevent view obstruction and minimize weight. To preserve the subject's visual field, an IR mirror can be placed in front of their eye enabling visible wavelengths to pass through while directing the emitted IR beam to the detector outside their field of view. For example, in the case where the IR detectors are mounted to a frame with a glasses-like form factor, the IR mirror can be integrated within the cover or lens located in front of each of the wearer's eyes. The IR detector and source are connected to their respective modulation hardware via wire threaded through a commutator ensuring all bulky components are kept separate from the animal. Embodiments of our device meant for humans deliver light to the pupils while minimally disturbing the user. To this end, the IR source is installed either in the user's ear canals, nostrils, or flush with their skin at a favorable angle.

The optical system is connected to an electronic controller placed away from the subject. We have already implemented the signal processing device with an Arduino, using transistor amplifiers to drive the LED with enough current, and with operational amplifiers to pick up weak currents from the photodiodes.

### Preliminary Results

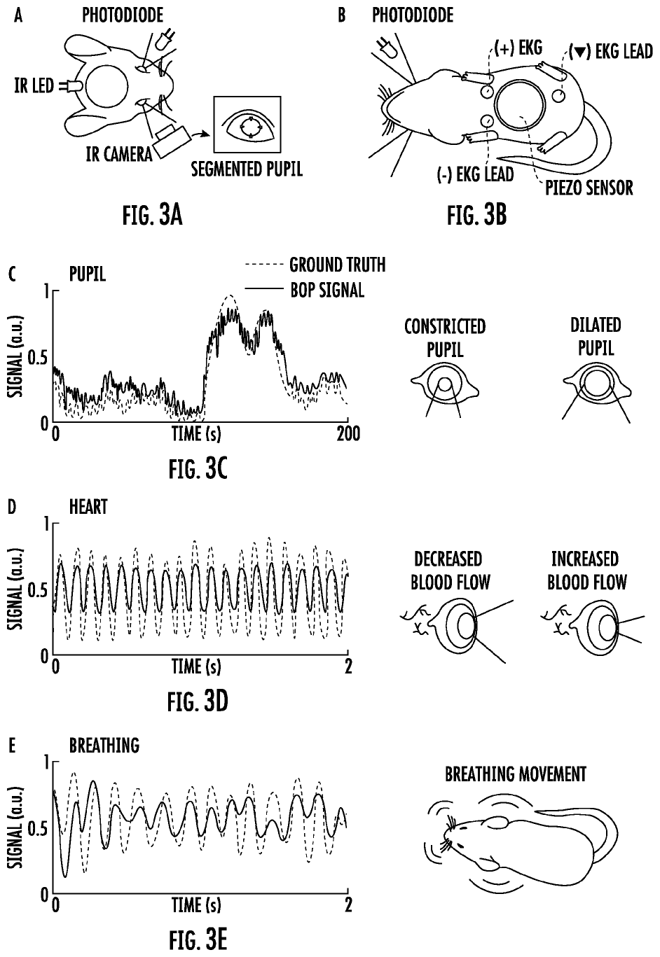

We have tested our first prototype with an IR led placed on the skull of a restrained mouse, and by placing the photodetector near the eye. For validation purposes, we recorded data with our invention while simultaneously recording video data of the pupil with a fast camera. An example test setup used to validate pupil size, heart beat, and respiration signals generated using the output from the biometric optical photometer is shown in FIGS. 3A and 3B. In FIG. 3A, the eyes of the mouse are back illuminated using a biometric optical photometer including an infrared LED and a photodiode, which detects the light exiting the pupils of the mouse. An infrared camera positioned adjacent to and pointed at one of the mouse's pupils is used to record images of the pupil from which ground truth pupil size data is generated. An EKG whose leads are illustrated in FIG. 3B produces a ground truth heart rate or heart beat signal. A piezo sensor mounted on the back of the mouse in FIG. 3B produces a ground truth signal of individual respirations.

Experimental results show that our technique successfully measures pupil size and that our experiments (shown by the solid lines in FIGS. 3C-3E) match ground truth data (shown by the dashed lines in FIGS. 3C-3E).

In particular, FIG. 3C shows that pupil sized measurements generated from low-frequency filtered biometric optical photometry (BOP) data match experimental recordings of pupil size measured from images produces by the infrared camera. Alongside eye motion, one benefit of our technology is that photodetectors enable extremely high sampling rates, orders of magnitude faster than video cameras. Fast sampling rates permit the detection of fast fluctuations of the photon flow through the pupil. This includes blinking, but also eye saccades, blood flow (pulse) and breathing rates. We demonstrate that individual heart beats can be resolved, and that processing our signal with a high pass filter, enables the detection of the animal heart's rate. We validated our data by recording our signal while the animal is connected to an EKG sensor. We found that individual heart cycles recorded from our device (shown by the solid line in FIG. 3D) can be resolved and correlated with ground truth data (shown by the dashed line in FIG. 3D). In particular, FIG. 3D illustrates that heartbeat measurements generated from bandpass-filtered BOP data match EKG oscillations corresponding to individual heart beats of an anesthesized mouse. FIG. 3E illustrates that respiration measurements generated from bandpass filtered BOP data match piezo sensor pressure oscillations corresponding to individual breathing motions of an anesthesized mouse.

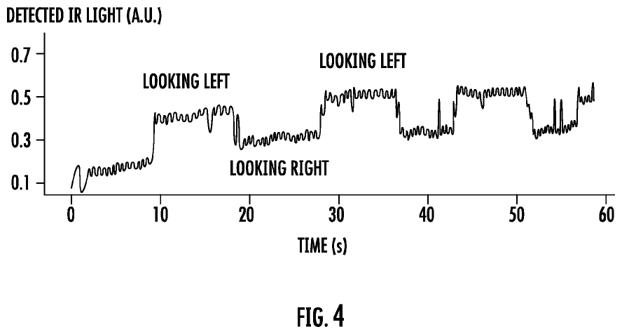

Preliminary results for the implementation of our device as wearable glasses (as in FIG. 2B) are shown in FIG. 4. With an IR source placed inside the ear, and a single photodiode placed near the eye, our data indicates clear variations of the collected flow of photons. When looking left, towards the photodetector, more photons are collected than when looking right, away from the photodetector. Aggregating similar data across several photodiodes will enable the tracking of the eye along with the measurement of additional ocular metrics, as detailed above.

### Electronic Interface

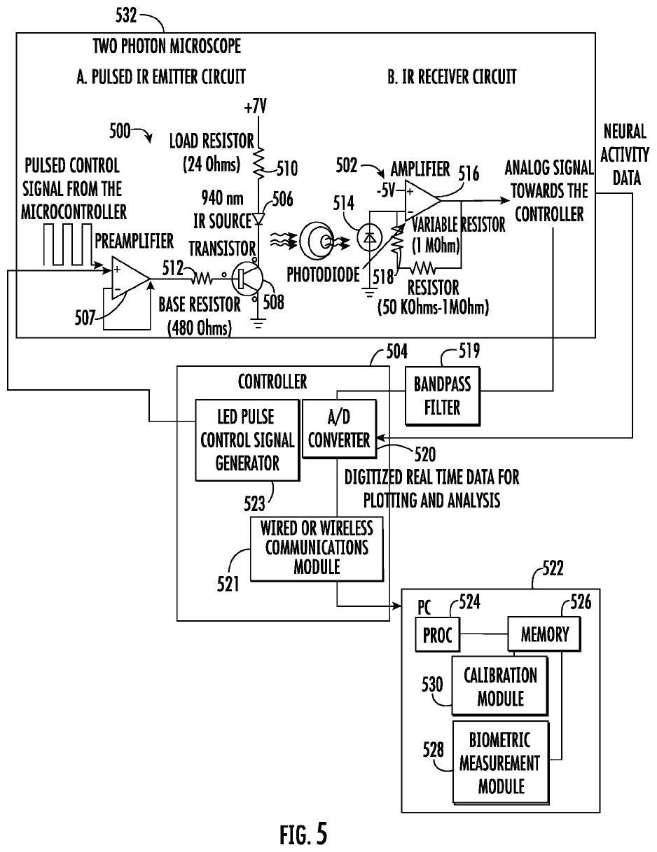

The electronic circuits for IR light pulsing and synchronous acquisition of the photon flux through pupils are shown in FIG. 5. More particularly, FIG. 5 illustrates an example of an infrared emitter circuit 500 used to implement each light source 100, an infrared receiver circuit 502 used to implement each light sensor 106, and a controller 504 for controlling the emitter and receiver circuits, digitizing and storing data generated by infrared receiver circuit 502, performing calibration, and generating output indicative of physical characteristics and activities of the subject.

Emitter circuit 500 includes an infrared source 506, which in the illustrated example is an infrared light emitting diode (LED) configured to emit infrared light at a wavelength of 940 nm. Emitter circuit 500 further includes an operational amplifier 507 configured as a voltage follower circuit to amplify and follow the pulsed control signal generated by controller 504. Emitter circuit 500 further includes a transistor 508 and associated resistors 510 and 512. Transistor 508 also functions as an amplifier, which amplifies the current passing through light source 506. In one example, controller 504 is capable of driving light source to generate pulses of ultrasound light in the kilohertz range, for example at a frequency in a range from 10 to 50 kilohertz at instantaneous power levels ranging between 1 milliwatt and 10 Watts.

Receiver circuit 502 includes an optoelectronic device, which in the illustrated example is a photodiode 514 for generating a current proportional to the intensity of detected light from emitter circuit 500 after exiting through the pupil of the subject. In an alternate example, the optoelectronic device may be implemented using another type of photodetector, such as a phototransistor. Receiver circuit 502 also includes an operational amplifier 516 that converts the photocurrent captured by the photodiode 514 into a voltage signal. A variable resistor 518 allows for fast adjustment of the gain of the current input to operational amplifier 516.

The output of operational amplifier 516 is an analog signal that is synchronously sampled by controller 504. A bandpass filter 519 may be included to filter the output along signal from operational amplifier 516 to remove the baseline (DC) component from the signal. Controller 504 includes an analog to digital (A/D) converter 520 which digitizes the signal and provides real time digitized data indicative of the detected light for plotting and analysis via a wired or wireless communications module 521 to a personal computer (PC) 522. Wired or wireless communications module 521 may be a wireless communications chip, such as a Wi-Fi, BlueTooth, or ultrawideband chip or a wired communications chip, such as an Ethernet or universal serial bus (USB) chip. Controller 504 also includes an LED pulse control signal generator 523 for generating the signal for controlling pulses generated by light source light source 506 synchronously with the sampling of the signal output by receiver circuit 502.

PC 522 includes at least one processor 524 and a memory 526. A biometric measurement module 528 reads the data received from controller 504 and generates real time data plots and output indicative of a physical characteristic or activity of the subject. A calibration module 530 performs the steps described herein for calibrating biometric measurement module 528, for example, to generate output indicative of light exiting the subject's eyes independently of the ambient light level.

In one optional configuration, emitter circuit 500 and receiver circuit 502 may be components of a two-photon microscope 532, which scans the brain of a subject using two infrared lasers to generate neural activity data. In such an implementation, controller 504 may be configured to record the indication of the light exiting the subject through the eye of the subject synchronously with neural imaging data produced by two-photon microscope 532.

In one example, biometric measurement module 528 may calculate an arousal index for a subject. Biometric ocular photometry allows gathering of multiple biometrics: pupillary, heart, respiratory dynamics. It is possible that an overarching latent arousal variable is a better metric than any of these metrics, individually. Therefore, we will develop an arousal-index (latent construct) designed to extract and integrate the most informative components of arousal garnered from the simultaneously acquired biometrics. For each individual arousal biometric, a unique function will be identified that maps that biometric into a lower dimensional latent space that fully expresses the temporal dynamics of that biometric. This function will be implemented using a 1-dimensional convolutional or recurrent neural network that is trained on the captured data from multiple subjects. This function is referred to as the encoder. Theoretically, this mapping should be identical across different subjects, therefore the data recorded from all subjects can be used to train the parameters of this mapping. The latent space for each biometric will be separable into a subspace that corresponds to an arousal index, and another complementary subspace describing the biometric specific dynamics. The arousal signal predicted by different encoders should reflect the same value and during the training of the encoder this subspace is shared and enforced to be equal for different encoders. Since the latent variables cannot be explicitly defined or experimentally measured, the encoder cannot be trained with supervised learning methods. Instead, we will use unsupervised algorithms to train our neural networks. To enable unsupervised training, we will use a separate network, termed decoder, that will predict the exact values of each biometric from the latent space corresponding to that biometric. The unsupervised loss will be calculated by quantifying the mismatch between the predictions of decoder and the ground truth biometric measurements and used to train both the encoder and the decoder.

Emitter and receiver circuits 500 and 502 are simultaneously controlled by controller 504 to synchronize digital pulsing of the IR light and analog recordings of the photocurrent in the photodiode(s). Receiver circuit 502 is replicated for each photodiode in the circuit, and the emitter circuit 500 is replicated for each desired illumination wavelength.

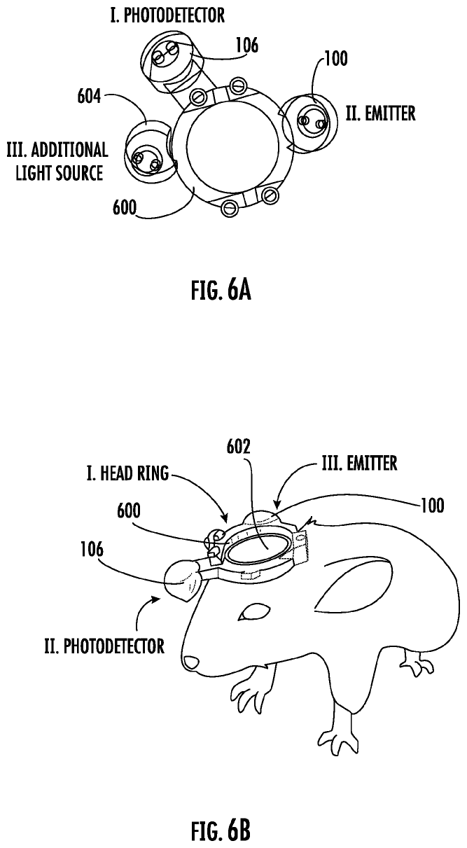

FIGS. 6A and 6B illustrate another example that includes a head mountable device for mounting the light sources and detectors on the head of an animal, such as a mouse. Referring to FIG. 6A, light sources 100 and light sensors 106 are mounted to an annulus 600. In one example, annulus 600 is configured to be mechanically attached to (e.g., clipped or bolted onto) a head plate 602 cemented to the skull of a mouse, as illustrated in FIG. 6B. Annulus 600 can be sized to fit the skull of larger or smaller subjects and can be configured to hold light sources, light sensors, or other devices at desired locations around the head of the subject. One example of another device that can be mounted to annulus 600 is a visible secondary light source 604 that allows the user to vary ambient light levels on demand to actively change the animal's pupil size. In such a configuration, light sources 100 would be considered the primary light sources.

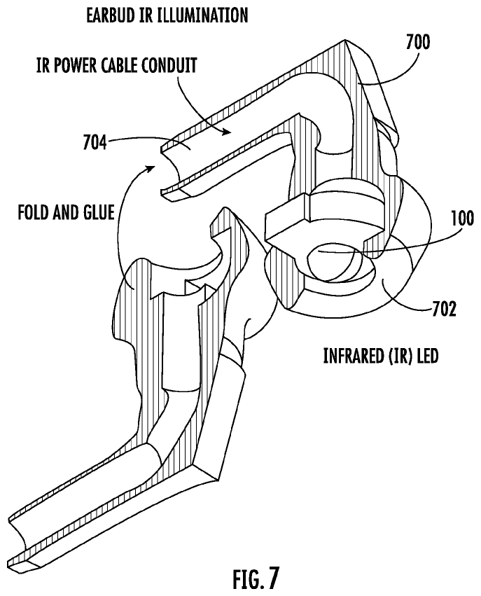

FIG. 7 illustrates an example where light source 100 is enclosed within a housing designed to fit within the ear canal of a subject. Referring to FIG. 7, light source 100 is enclosed within a housing 700, which has an ear-bud-like form factor designed to fit within the ear canal of a human subject. In the illustrated example, housing 700 includes an ear-insertable portion 702 that holds light source 100 and is shaped to fit within the ear canal of the subject. Housing 700 further includes a conduit 704 for enclosing power and/or signaling cables.

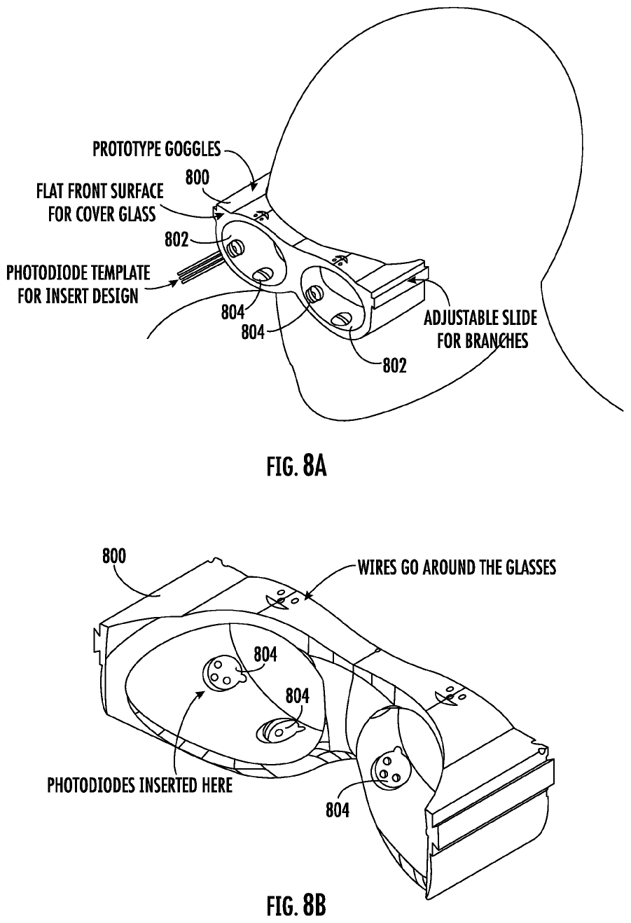

FIGS. 8A and 8B illustrate an example of a housing for holding light sensors 106 where the housing has a goggles-like form factor. In FIGS. 8A and 8B, housing 800 comprises a pair of goggles designed to be worn on the subject's head. Housing 800 includes eye opening portions 802, each of which includes a plurality of circumferentially spaced receptables 804 designed to hold light sensors 106. Receptables 804 are angled so that light sensors 106 will point towards a user's eyes to detect light from the light source exiting the eyes through the pupils.

### Potential Users

Our portable high-speed inexpensive arousal monitoring device has applications in medicine, research, marketing, and entertainment and will be of interest to a broad range of users. Manufacturers of miniature acquisition or photostimulation hardware can easily enhance their existing products with our device, gaining far more functionality with little additional cost. Open source lab instrumentation companies may also be interested in making the detection devices described herein. Rapid biometric monitoring is key to employ cutting edge neuroscience research which may entice major manufacturers of behavioral instrumentation equipment. Another application of the subject matter described herein is pupillometry and eye tracking for video games.

### Use Cases

The following are potential applications of biometric optical photometry as described herein:

### Head-Fixed Biometric Ocular Photometer

In this implementation of the invention, the BOP LED and sensors are held in a static position with respect to a head-fixed animal, restrained to an experimental setup. The LED, sensors, and other optical components are brought individually or together at their appropriate location. Each component is then wired to the controller.

### Freely Moving Biometric Ocular Photometry

In this implementation, the optical components of the BOP are assembled on a small 3D printed frame that is surgically glued to the skull of the animal. After recovery, the implanted device is connected to the control board by means of a wire tether allowing free displacements within the range of the experimental assay. This implementation can be modified to be compatible with other neuroscience technologies that target neural ensembles (i.e. optogenetics, 1 photon miniscopes). The geometry of the head implant can be adjusted to leave clear access to specific brain regions to implement these other modalities.

### Wireless Biometric Ocular Photometry

In this implementation, the optical components of the BOP are assembled on a small 3D printed frame and connected to a circuit board that is surgically glued to the skull of the animal. The circuit board includes a wireless chip (e.g., a Wi-Fi, BlueTooth, or ultrawideband chip), control electronics, signal recording and digitization, as well as a small detachable battery. Once the battery is in place, the circuit can be turned on and controlled remotely, usually via bluetooth protocols. This modality allows experimental recordings from freely-moving, untethered, animals.

### Two-photon Biometric Ocular Photometry

This implementation enables the BOP signal and calcium activity from neural ensembles to be captured concurrently. By routing the signal from the photodiode to a channel that is normally connected to one of the two photomultiplier tubes (PMT) in a 2 photon microscope, BOP data can be read in synchronously with neural activity, all within the same data acquisition system. The data is then processed to extract biometric information. In this modality, the infrared laser used for two-photon imaging also serves as an illumination source for BOP measurements. This modality involves an additional computational step to compensate for the scanning of the laser beam over the imaged brain region.

### Eye Tracking Biometric Ocular Photometry, Both for Headsets and Implants

In this implementation, the optical components of BOP and multiple sensors are used to track the position of the pupil in real-time. Differences in intensity collected from each sensor are used to infer pupil position along the horizontal and vertical directions. This implementation can be added to any other modality.

### Biometric Ocular Photometry as an Eye-Tracking Device Only

The technology can be utilized for the sole purpose of tracking eye motion in substitution for any currently used eye tracking technology. Eye tracking is used in behavioral studies, for user experience feedback, or in consumer applications to control a touchless screen. It can also be used as an assistive device for patients suffering from heavy paralysis and rely on eye-controlled keyboards to communicate.

### Biometric Ocular Photometry for Pupillometry Through a Closed Eye Lid

The BOP technology can be used to monitor pupil size through a closed eyelid. The eye lid adds a static amount of absorption, but infrared light can still be detected through it. Operating a BOP sensor through a closed eye lid continues to capture fluctuations of the pupil size. Hence, this modality can be used to measure the pupillary reflex in sleep studies or during surgery, to estimate the level of awareness (e.g. patient is awake) in real time.

### Features

The following are features of the subject matter described herein.

Back-illumination. Instead of recording signals without a light source or with light originating from the front of the face, our approach illuminates the back of the eye using diffused light passing through bone, skin and tissue, enabling the collection of hemodynamics similar to pulse oximeters. The wavelength we operate with is selected to allow maximal transmission through tissue and is not detected by the human eye.

Camera-free technique. Many pupillometry techniques measure the size of the eye from camera images. Our camera-free design facilitates portability. Cameras are slower than our technique and generate large datasets that need substantial processing. Our device outputs analog signals, achieves kilohertz sampling speeds, and does not require substantial processing to live-stream the raw data in real-time.

Simultaneous parallel acquisition of eye and body biometrics and environmental cues. Our technique not only measures eye properties such as motion and pupil size, but also body metrics (heart and breathing rate, and properties of the environment (ambient light, head motion). All existing technologies only allow a fraction of these measurements and from distinct devices.

Continuous tracking across changing environments of pupillary dynamics and markers of arousal. Our invention does not reduce the field of view and relies on invisible light that the eye cannot detect. Low power operation also allows for continuous and comfortable operation for several hours and without disturbing behavior.

Classification of pupillary events. Since our device simultaneously captures both the level of ambient light, and pupil size in real time, our algorithm can reliably identify if any detected pupillary change results from either an environmental factor, i.e., a variation in the ambient light, or if the event occurs in response to behavioral changes or arousal responses.

Fashionable device with a compact form factor. Our technology can be integrated within safety equipment, or as a standalone modified pair of glasses. The detectors can be easily concealed in a pair of glasses' frame, and the IR source can be built as a small ear-plug or a skin-contact device placed at the edge of the glasses. The device can be made both comfortable for implementation in personal protective equipment, and as a fashionable device for consumer-grade utilizations.

Head mountable device for research in freely moving animals. Our technology employs a low weight device that allows animals to still engage in their full repertoire of naturalistic behavior, as opposed to heavier technologies that employ bulky camera systems.

Self-calibrating algorithms with real time operation. We will implement machine learning models trained offline with phantoms to directly extract the relevant data from the raw signal measured by the sensors. This strategy has the advantage to merge calibration and processing into the same pipeline and facilitate the utilization of the same device on various people and/or animal models.

Compatibility with existing neuroscience technology. Our system can be used alongside other optical brain interfacing technology including calcium, voltage imaging methods as well as optogenetic techniques, allowing its implementation as an add-on to many existing experimental protocols, and without disturbing their respective operations.

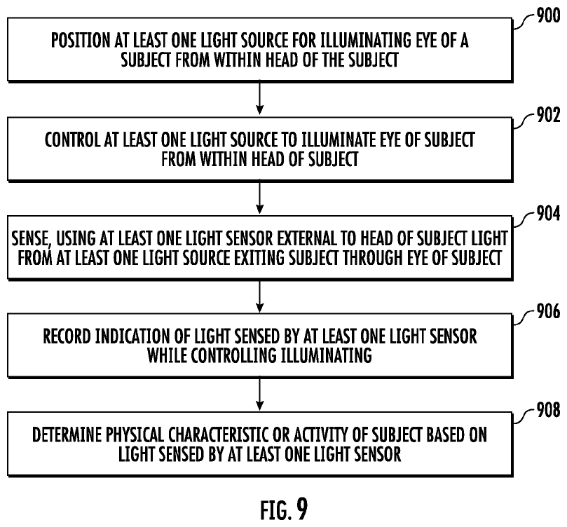

FIG. 9 is a flow chart illustrating an exemplary process for measuring a physical characteristic or activity of a subject using back-illumination of an eye of the subject. Referring to FIG. 9, in step 900, the process includes positioning at least one light source for illuminating an eye of a subject using light from within a head of the subject. For example, one or more light sources 100 may be positioned within the head of a subject in an ear canal of the subject, within a nostril of the subject or against the subject's skin to shine light into the head of the subject.

In step 902, the process includes controlling the at least one light source to illuminate the eye of the subject from within the head of the subject. For example, controller 504 may control light source(s) 100 to generate pulses of infrared light to illuminate the eye of the subject from within the subject's head.

In step 904, the process includes sensing, using at least one light sensor located external to the head of the subject, light from the at least one light source exiting the subject through the eye of the subject. For example, one or more sensors 106 may detect light exiting the subject's eyes through the pupils of the subject's eyes. It should be noted that the sensing performed in this step can detect light exiting the subject's eyes when the subject's eyes are open or closed. That is, if a subject is sleeping or undergoing anesthesia, for example, sensors 106 may detect light exiting the subject's eyes through the subject's closed eyelids, which may be partially transparent to some wavelengths of light, such as infrared light wavelengths.

In step 906, the process includes recording an indication of the light sensed by the at least one sensor while controlling the illuminating. For example, controller 504 may digitize and record data detected by sensors 106 as they detect light exiting the user's eyes. The recording may be performed synchronously with the pulsing of light by light source 100.

In step 908, the process includes determining a physical characteristic or activity of the subject based on the light detected by the light sensors. For example, the biometric measurement module may determine at least one of eye movement, eye blinking, pupil size variation, heart rate, heart beats, blood flow, pulse oximetry, breathing rate, and respirations of the subject based on the light sensed by the at least one light sensor.

The disclosure of each of the following references is incorporated herein by reference in its entirety.

### REFERENCES

1. Mathot, S. Pupillometry: Psychology, Physiology, and Function. J. Cogn. 1, 16 (2018).
2. Hall, C. A. & Chilcott, R. P. Eyeing up the Future of the Pupillary Light Reflex in Neurodiagnostics. Diagnostics 8, 19 (2018).
3. Fried, R. Pupillometry: The Psychology of the Pupillary Response (Book). J. Pers. Assess. 44, 441-444 (1980).
4. Kahya, M. et al. Pupillary Response to Postural Demand in Parkinson's Disease. Front. Bioeng. Biotechnol. 9, 617028 (2021).
5. Mathôt, S., Melmi, J.-B., van der Linden, L. & Van der Stigchel, S. The Mind-Writing Pupil: A Human-Computer Interface Based on Decoding of Covert Attention through Pupillometry. PLOS ONE 11, e0148805 (2016).
6. Mitre-Hernandez, H., Covarrubias Carrillo, R. & Lara-Alvarez, C. Pupillary Responses for Cognitive Load Measurement to Classify Difficulty Levels in an Educational Video Game: Empirical Study. JMIR Serious Games 9, e21620 (2021).
7. Rodriguez-Romaguera, J. et al. Prepronociceptin-Expressing Neurons in the Extended Amygdala Encode and Promote Rapid Arousal Responses to Motivationally Salient Stimuli. Cell Rep. 33, 108362 (2020).
8. Reimer, J. et al. Pupil fluctuations track fast switching of cortical states during quiet wakefulness. Neuron 84, 355-362 (2014).
9. Privitera, M. et al. A complete pupillometry toolbox for real-time monitoring of locus coeruleus activity in rodents. Nat. Protoc. 15, 2301-2320 (2020).
10. Roth, N. Automatic Optometer for Use with the Undrugged Human Eye.
Rev. Sci. Instrum. 36, 1636-1641 (1965).
11. Meyer, A. F., Poort, J., O'Keefe, J., Sahani, M. & Linden, J. F. A Head-Mounted Camera System Integrates Detailed Behavioral Monitoring with Multichannel Electrophysiology in Freely Moving Mice. Neuron 100, 46-60.e7 (2018).
12. Kim, S.-Y. et al. Diverging neural pathways assemble a behavioural state from separable features in anxiety. Nature 496, 219-223 (2013).
13. Stiedl, O. & Spiess, J. Effect of tone-dependent fear conditioning on heart rate and behavior of C57B L/6N mice. Behav. Neurosci. 111, 703-711 (1997).
14. Sjoding, M. W., Dickson, R. P., Iwashyna, T. J., Gay, S. E. & Valley, T. S. Racial Bias in Pulse Oximetry Measurement. N. Engl. J. Med. 383, 2477-2478 (2020).
15. Craske, M. G. et al. Anxiety disorders. Nat. Rev. Dis. Primer 3, 17024 (2017).
16. Lang, P. J. & McTeague, L. M. The anxiety disorder spectrum: fear imagery, physiological reactivity, and differential diagnosis. Anxiety Stress Coping 22, 5-25 (2009).
17. Wilhelm, F. H. & Roth, W. T. The somatic symptom paradox in DSM-IV anxiety disorders: suggestions for a clinical focus in psychophysiology. Biol. Psychol. 57, 105-140 (2001).
18. Ortiz-Juza, M. M., Alghorazi, R. A. & Rodriguez-Romaguera, J. Cell-type diversity in the bed nucleus of the stria terminalis to regulate motivated behaviors. Behav. Brain Res. 411, 113401 (2021).
20. Pegard, N. et al. Holographic Temporal Focusing for 3D Photo-activation With Single Neuron Resolution. Nat. Comm., 2017, 3-5 (2017).
21. Mardinly, A. R. et al. Precise multimodal optical control of neural ensemble activity. Nat. Neurosci. (2018) doi: 10.1038/s41593-018-0139-8.
22. Eybposh, M., Caira, N., Atisa, M., Chakravarthula, P. & Pegard, N. DeepCGH: 3D Computer-Generated Holography Using Deep Learning. Opt. Express (2020) doi: 10.1364/oe.399624.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A system for determining a physical characteristic or measuring a physical activity of a subject using back-illumination of an eye of a subject, the system comprising:
at least one light source configured for illuminating an eye of a subject using light from within the head of the subject;
at least one light sensor positionable outside of the head of the subject for sensing light from the at least one light source exiting the subject through the eye of the subject; and
a controller coupled to the at least one light source and to the at least one light sensor for recording an indication of the light while controlling the illuminating.

2. The system of claim 1 wherein the at least one light source comprises an infrared light source.

3. The system of claim 2 wherein the infrared light source comprises an infrared emitter circuit configured to generate pulses of infrared light.

4. The system of claim 3 wherein the infrared emitter circuit is configured to generate continuous illumination of pulses of light at instantaneous power levels ranging from 1 milliwatt to 10 Watts.

5. The system of claim 1 wherein the at least one light source comprises a housing configured to be inserted into an ear canal or a nostril of the subject or positioned adjacent to a surface of the skin of the subject.

6. The system of claim 1 wherein the at least one light source comprises an implantable device for implantation within the head of the subject.

7. The system of claim 1 wherein the at least one light source comprises a primary light source for illuminating the eye of the subject from within the head of the subject and a secondary light source for controlling a pupil of the eye of the subject by illuminating the pupil externally to the head of the subject.

8. The system of claim 7 wherein the secondary light source comprises a visible light source.

9. The system of claim 1 comprising a head mountable frame for holding the at least one light source.

10. The system of claim 9 wherein the head mountable frame is configured to hold the at least one light sensor.

11. The system of claim 9 wherein the head mountable frame has a goggles-like or a glasses-like form factor.

12. The system of claim 9 wherein the head mountable frame comprises an annulus for holding the at least one light sensor near the eye of the subject.

13. The system of claim 1 comprising a mirror for directing the light exiting the subject through the eye of the subject to the at least one light sensor.

14. The system of claim 13 wherein the at least one light source comprises an infrared light source and the mirror comprises an infrared mirror.

15. The system of claim 1 wherein the at least one light sensor comprises a light sensor array including a plurality of infrared receiver circuits positionable around an exterior of the eye of the subject.

16. The system of claim 15 wherein the infrared receiver circuits each comprise an optoelectronic device for detecting the light and generating a current proportional to the detected light.

17. The system of claim 16 wherein the optoelectronic device comprises a photodetector.

18. The system of claim 1 wherein the controller includes an analog-to-digital converter for producing digital values based on a signal generated by the at least one light sensor.

19. The system of claim 1 wherein the controller records at least one signal generated by the at least one light sensor while synchronously controlling the at least one light source to generate pulses of light.

20. The system of claim 1 comprising a biometric measurement module for determining a physical characteristic of the subject based on the light sensed by the at least one light sensor.

21. The system claim 20 wherein the physical characteristic determined by the biometric measurement module includes an indicator of at least one of eye movement, eye blinking, pupil size variation, and an arousal state of the subject.

22. The system of claim 20 wherein the physical characteristic determined by the biometric measurement module includes at least one of heart rate, heart beat, blood flow, pulse oximetry, breathing rate, and respirations of the subject.

23. The system of claim 20 comprising a calibration module that implements an algorithm configured to calibrate the biometric measurement module.

24. The system of claim 20 wherein the biometric measurement module is configured to track the eye of the subject based on the light sensed by the at least one light sensor.

25. The system of claim 1 comprising a two-photon microscope, wherein the at least one light source and the at least one light sensor comprise components of the two-photon microscope and the controller is configured to record the indication of the light exiting the subject through the eye of the subject synchronously with neural imaging data produced by the two-photon microscope.

26. The system of claim 1 wherein the controller includes a wired or wireless communications module for communicating the indication of the light exiting the subject through the eye of the subject to a computing platform over a wired or wireless connection.

27. The system of claim 1 wherein the controller is configured to subtract a representation of a signal generated by the at least one light sensor when the at least one light source is off from a representation of the signal generated by the at least one light sensor when the at least one light source is on for measuring an amount of light exiting the subject through the eye of the subject regardless of ambient light levels.

28. The system of claim 1 wherein the at least one light sensor is configured to sense the light exiting the subject through a pupil of the eye of the subject.

29. The system of claim 1 wherein the at least one light sensor is configured to sense the light exiting the subject through a closed eyelid of the subject.

30. A method for determining a physical characteristic or measuring a physical activity of a subject using back-illumination of an eye of the subject, the method comprising:
positioning at least one light source for illuminating an eye of a subject using light from within the head of the subject;
controlling the at least one light source to illuminate the eye of the subject from within the head of the subject;
sensing, using at least one light sensor located external to the head of the subject, light from the at least one light source exiting the subject through the eye of the subject; and
recording an indication of the light sensed by the at least one light sensor while controlling the illuminating.

31. The method of claim 30 wherein positioning the at least one light source includes positioning the at least one light source in an ear canal, in a nostril, or adjacent to a surface of the skin of the subject.

32. The method of claim 30 wherein positioning the at least one light source includes positioning the at least one light source within or on the head of the subject.

33. The method of claim 30 wherein recording the indication of the light sensed by the at least one light sensor while controlling the illuminating includes recording at least one signal generated by the at least one light sensor while synchronously controlling the at least one light source to generate pulses of light.

34. The method of claim 30 comprising determining a physical characteristic or activity of the subject based on the light sensed by the at least one light sensor.

35. The method of claim 34 wherein the physical characteristic or activity includes at least one of eye movement, eye blinking, and pupil size variation based on the light sensed by the at least one light sensor.

36. The method of claim 34 wherein the physical characteristic or activity includes at least one of heart rate, heart beats, blood flow, pulse oximetry, arousal state, breathing rate, and respirations of the subject based on the light sensed by the at least one light sensor.

37. The method of claim 34 comprising subtracting a representation of a signal generated by the at least one light sensor when the at least one light source is off from a representation of the signal generated by the at least one light sensor when the at least one light source is on for measuring an amount of light exiting the subject regardless of ambient light levels.

38. The method of claim 30 wherein sensing the light exiting the subject through the eye of the subject includes sensing the light exiting the subject through a pupil of the eye of the subject.

39. The method of claim 30 wherein sensing the light exiting the subject through the eye of the subject includes sensing the light exiting the subject through a closed eyelid of the subject.

40. The method of claim 30 comprising tracking the eye of the subject based on the light sensed by the at least one light sensor.

41. The method of claim 30 comprising recording neural imaging data output from a two-photon microscope synchronously with the recording of the indication of the light exiting the subject through the eye of the subject.

42. The method of claim 30 comprising communicating the indication of the light exiting the subject through the eye of the subject to a computing platform over a wired or wireless connection.

43. A non-transitory computer readable medium having stored thereon executable instructions that when executed by a processor of a computer control the computer to perform steps comprising:
controlling at least one light source to illuminate an eye of a subject from within the head of the subject;
sensing, using at least one light sensor located external to the head of the subject, light from the at least one light source exiting the subject through the eye of the subject; and
recording an indication of the light sensed by the at least one light sensor while controlling the illuminating.
